(19) United States
(12) Patent Application Publication
(10) Pub. No.: US 2022/0290096 A1
(43) Pub. Date: Sep. 15, 2022

(54) METHOD FOR PRODUCING BIOLOGICAL TISSUE

(71) Applicant: AXENOLL LIFE SCIENCES AG, Zurich (CH)

(72) Inventors: David L. Deck, Zurich (CH); Srdan Vasic, Zurich (CH)

(21) Appl. No.: 17/628,122

(22) PCT Filed: Jul. 16, 2020
(86) PCT No.: PCT/EP2020/070222

§ 371 Date: Jan. 18, 2022

(30) Foreign Application Priority Data
Jul. 18, 2019 (EP) 19187072.4

(51) Int. Cl. C12N 5/00; B33Y 80/00

(52) U.S. Cl. CPC C12N 5/0062; B33Y 80/00; C12N 2513/00; C12N 2535/00

(57) ABSTRACT

Method for producing biological tissue, in particular for medical treatment and/or pharmacological examinations, wherein a support for cell tissue is provided, wherein a printing medium containing living cells is provided, wherein the printing medium is printed onto the support through a printing screen and/or a printing stencil, and wherein the printed cells develop into tissue by the printing and/or after the printing.

### CROSS REFERENCE TO RELATED APPLICATIONS

This application is a U.S. National Stage of International Patent Application No. PCT/EP2020/070222 filed Jul. 16, 2020, which claims the benefit of priority of European Patent Application No. EP19187072.4 filed Jul. 18, 2019, the respective disclosures of which are each incorporated herein by reference in their entireties.

### FIELD OF THE INVENTION

The invention relates to a method for producing biological tissue. Likewise, the invention relates to biological tissue produced according to such a method.

### BACKGROUND OF THE INVENTION

It is known that tissue can be produced, for example, by inkjet printing. Printing media with living cells can be used for this purpose. However, this can regularly expose the cells to high mechanical stresses. Depending on the design of the printing systems, the cells can be subjected in particular to high shear and/or pressure loads and/or frictional loads. The survival rate of the cells can be reduced as a result and the cell tissue produced in each case can thus be impaired.

Furthermore, inkjet printing of cells or of printing media with cells contained therein is associated with the disadvantage that 2-dimensional arrangements can only be built up sequentially. Three-dimensional printing by inkjet printing is therefore associated with even longer production times. Such long printing or production times impair cost-effectiveness. At the same time, long printing or production times can have a negative impact on the survival rate of the cells, since living cells can only divide to a limited extent. If several cell divisions have already taken place by the time an entire print is completed, the further survivability of the cells may be severely limited in time.

### SUMMARY

Against the above-mentioned background, it has been the object of the present invention to disclose a method for producing biological tissue that can be carried out with improved productivity while reducing the risk of cellular damage. Likewise, the object has been to disclose a tissue produced by such a method.

With regard to the method, this object has been solved with the subject matter of claim 1 and a tissue according to the invention is subject to claim 15. Advantageous embodiments are the subject of the dependent claims and explained hereinafter.

In a method for producing biological tissue according to the invention, a support for cell tissue is provided, a printing medium containing living cells is provided, the printing medium is printed onto the support through a printing screen and/or a printing stencil, and the printed cells develop into tissue as a result of the printing and/or after the printing.

Tissue generated by a method according to the invention is particularly suitable for conducting medical and/or pharmacological examinations and/or treatments. In particular, tissue generated by a method according to the invention can be used for in vitro examinations, preferably for analytical purposes, and/or for in vivo treatments, preferably for medical purposes.

By printing the printing medium through a printing screen and/or a printing stencil, a high degree of productivity can be ensured in an advantageous manner. In particular, the design of the printing screen and/or printing stencil permits the printing of defined printing areas or entire printing layers and/or tissue layers in just one printing step. The entire printing process can thus be completed in only a short period of time. The remaining survival period of the respective cells can thus be extended.

At the same time, the printing screen or printing stencil can advantageously reduce the shear stresses, pressure stresses and/or frictional stresses of the cells in the printing medium. Openings of the printing screen and/or the printing stencil can be designed in sufficient size with regard to reduced mechanical stresses on the cells, such as shear stresses, pressure stresses and/or frictional stresses. The risk of damage to the cells in the printing medium as a result of the process of printing or application to the support can thus be reduced. The suitability of the tissue produced according to the invention for medical treatments and/or pharmacological examinations is thereby improved. In particular, printing the printing medium containing living cells through a printing screen and/or through a printing stencil onto a support enables the particularly advantageous development of the printing medium into tissue.

In the present context, tissue is understood to mean cell tissue, in particular biological tissue consisting of cells and/or containing cells.

A support can be understood here as a machine-based support or a support provided, in particular fixedly provided, on a printing system. Likewise, a support can be a carrier structure that is positioned in a printing system only for a printing process or until printing is completed and is then removed from the respective printing system together with the printed object. The carrier structure can be, for example, an object carrier and/or a cell culture plate. Such a carrier structure is particularly advantageously suited for use and/or handling in laboratories or for standardized transport containers, transport devices and/or storage devices.

In terms of the present invention, a printing screen may be provided with a fabric or formed by a fabric and/or by a frame for the fabric. Such a fabric may be configured for printing a printing paste therethrough. The fabric of a printing screen may be arranged or clamped in a frame. Further, a fabric may be formed by metal and/or plastic threads. Such metal and/or plastic threads can be arranged twisted or run individually.

To achieve the desired printing shape in each case, a printing screen can have permeable and impermeable areas. Permeable regions may be permeable to a printing medium and impermeable regions may be impermeable to a printing medium. Permeable regions of a printing screen, in terms of the present invention, may form a passage and/or a cutout in which a fabric permeable to a printing medium is disposed. Impermeable regions may also be provided with a fabric, which, however, is designed to be impermeable to a printing medium or is covered to achieve impermeability.

With a printing screen fine structures, in particular with a thickness between 15 μm and 500 μm, can be produced. Printing by means of a printing screen can be carried out with relatively high accuracy, in particular with high accuracy in the boundary areas of the printed product.

Within the meaning of the present invention, a printing stencil may be provided with openings and/or cutouts, in particular with through-openings and/or cutouts made in a plate. A plate with openings and/or cutouts may form a printing stencil within the meaning of the present invention. Openings and/or cutouts may accordingly form a passage for a printing paste. Printing paste can be forced through such openings and/or cutouts as part of a printing process. In particular, a printing stencil can be formed free of fabric. In particular, the openings and/or cutouts of a printing stencil can be free of fabric.

At the same time, it is possible for a form element to be arranged within an opening and/or a cutout of a printing stencil in order to define the shape of the opening and/or the cutout in accordance with the respectively desired printing shape. Such form elements can be held in the desired position within the respective opening and/or cutout by means of individual holding threads or the like.

Printing stencils are particularly suitable for printing layers with a thickness from 100 μm, especially from 300 μm, and particularly preferably from 500 μm. Printing by means of a printing stencil can be carried out at a relatively high speed, since greater application thicknesses can be achieved.

According to a further advantageous embodiment, a carrier structure and/or support can be formed for the arrangement and/or receiving of a plurality of cell cultures and/or cell agglomeration and/or tissue structures separated from one another. Such a carrier structure can in particular have predefined arrangement and/or receiving sections for cell cultures and/or cell agglomeration and/or tissue structures separated from one another. This can ensure a secure and defined arrangement of the printed cells or cell cultures and/or cell agglomeration and/or tissue structures formed by the cells on the carrier structure.

In a preferred manner, the arranging and/or receiving sections may be provided in a plurality of lines and rows on the carrier structure and/or the support. In particular, the number of lines may be different from the number of rows. The outer shape of the carrier structure and/or support may be adapted to the number of rows and lines. Further preferably, the carrier structure and/or the support may have recesses for receiving the printing medium and/or the arrangement and/or receiving sections may be formed as recesses.

It is also possible for a carrier structure to be printed together with and/or alternately with the printing medium containing living cells and be thereby provided. This can ensure a high degree of manufacturing flexibility. The positioning and alignment of a finished carrier structure below a printing screen and/or below a printing stencil can thus be omitted. The preparation effort for printing can thus be reduced.

Similarly, it is possible for a carrier structure to be printed prior to printing the print medium containing living cells and to be provided in this way. The fully printed carrier structure can then be used as a printing substrate.

According to a further preferred embodiment, the printing medium may be printed through a printing screen onto the support with or without a scaffold, matrix and/or structural framework. Further, at least one structural framework, matrix and/or scaffold may be provided on the support. It is further possible for structural scaffolds, matrices, and/or scaffolds to be printed together with and/or contained within the printing medium. Scaffolds, matrices and frameworks are particularly advantageously suited for the generation of three-dimensional tissue structures. Cells, cell cultures and/or cell agglomerations can be formed three-dimensionally or developed into three-dimensional tissue structures in this way with only little effort and at the same time reproducibly.

When printing the printing medium containing living cells, cavities, recesses, linear or channel-shaped structures can be provided in a particularly advantageous manner. Such shapes allow improved functionalization of the cell cultures, cell agglomerations and/or tissues produced with the printing medium. For example, scaffolds and/or blood vessels and/or nerves or nerve tracts can be formed by such cavities, recesses and/or structures.

In a particularly preferred manner, a combination of materials can be printed, especially to produce frameworks and/or scaffolds and/or matrices with cells in a single printing process with only little effort.

It can be further advantageous if the print medium is printed in layers and/or built up in layers. Similarly, a single printing layer and/or printing layer can be produced in a single printing process and/or a printing layer can have a smaller thickness in the print build-up direction than the extent of the printing layer transverse to the print build-up direction. In this way, three-dimensional tissue structures can be produced with only little effort and at the same time with high accuracy.

According to a further advantageous embodiment, single-layer or multilayer cell agglomerations and/or single-layer or multilayer cell cultures and/or single-layer or multilayer tissues and/or tissue structures can be generated by printing the printing medium. The printing of a single-layer cell agglomeration and/or cell culture and/or tissue structure can be realized with only little effort and thus at low cost. Multi-layered cell agglomerations and/or cell cultures and/or tissue structures enable particularly meaningful examinations and/or particularly far-reaching medical treatments. The conditions of living organisms or the properties of human or animal tissue and/or organ structures can be particularly well reproduced by a multilayer design.

In a further preferred manner, a multilayer tissue or a multilayer tissue structure can be easily and quickly produced by printing a printing medium containing living cells several times and/or by varying the cells or the printing medium containing cells in a layer build-up direction (z-direction). Such a multilayer tissue can be, for example, skin or also other cell tissue types and/or organ structures.

In a further preferred manner, the printing medium can be fed through the printing screen and/or through the printing stencil and/or onto the support by at least one blading process and/or at least one printing blade movement, in particular by a plurality of blading processes and/or a plurality of printing blade movements. Blading operations or printing blade movements permit rapid and uniform printing of the printing medium through the printing screen or through the printing stencil. In particular, this allows particularly reproducible results to be achieved.

In a particularly preferred manner, the printing medium can be printed by at least one screen printing process, in particular by a plurality of screen printing processes. In particular, the printing of the print medium can be carried out by a screen printing process and/or stencil printing process, especially preferably by a 2D screen printing process or 3D screen printing process. This can ensure a particularly high level of productivity and accuracy. In the screen printing process, relatively large volumes of a printing medium can be printed with only small efforts of time and costs. The arrangement and/or generation of cell cultures and/or cell agglomerations and/or tissue or tissue structures can thus be carried out quickly and cost-effectively.

At the same time, the use of a screen printing process, in particular a 2D screen printing process or 3D screen printing process, ensures gentile processing of the printing medium containing living cells. The respective cells can be processed and/or printed in the screen printing process with only a low risk of damage due to shear stresses and/or pressure stresses and/or frictional stresses. In particular, a 3D screen printing machine can be used for printing the printing medium.

In a further preferred manner, substructures of the cell cultures and/or cell agglomerations and/or tissues and/or tissue structures are printed and/or produced with an accuracy of up to 100 cells, preferably up to 50 cells, in particular 20 cells, further preferably 10 cells, in particular down to a single cell. Such printing or manufacturing accuracies can be achieved both in the layer build-up direction (z-direction) and/or in directions transverse to the layer build-up direction (x/y-directions). In particular, such accuracies can be realized by means of 3D screen printing.

The screen printing process or the plurality of screen printing processes can advantageously be carried out on, in particular directly on, the support. Screen printing on a support, for example on a machine-based support and/or on a cell carrier or object carrier, enables a high degree of reproducibility and ease of handling when removing the printed object after the printing process is complete.

In a still further preferred embodiment, a three-dimensional cell culture and/or a three-dimensional cell agglomeration may be generated by printing the printing medium onto the support. After printing onto the support, the respective cell culture and/or cell agglomeration may develop into a three-dimensional tissue structure. Similarly, the three-dimensional tissue structure may already develop as a result of the printing of the printing medium. Accordingly, a development time may be required between the completion of printing and the development of a cell culture, cell agglomeration and/or tissue structure. Over the duration of such a development time, the properties and/or characteristics of the tissue structure, cell cultures and/or cell agglomerations can be influenced. Three-dimensional cell cultures and/or cell agglomerations and/or tissue structures can be produced in particular by repeated printing or by 3D screen printing.

According to a further preferred embodiment, it can be provided that the printed cells and/or cell agglomerations develop into organ tissue, in particular liver tissue, kidney tissue, heart tissue, skin tissue and/or muscle tissue, and/or into a partial or complete organ, in particular liver, kidney, heart and/or skin, preferably for transplantation, as a result of the printing or after printing. Such tissue structures are particularly advantageously suitable for medical treatments and/or pharmacological examinations.

In a further preferred manner, the printed cells may develop through the printing and/or after the printing into food grade tissue and/or food. In particular, the respective cell culture and/or cell agglomeration may develop through the printing and/or after the printing into synthetic meat that may be suitable for consumption. In particular, it is possible that the printed cells and/or cell agglomerations develop into a meat-containing food as a result of the printing or after the printing.

In a further preferred manner, the printing screen and/or the printing stencil can have at least one cutout, in particular several cutouts, for the passage of the printing medium, in particular cutouts formed separately from one another. A defined passage of the printing medium through the printing screen and/or the printing stencil can thus be realized. In particular, the printing medium can thus be printed in a defined manner at different positions and/or positions spaced-apart from each other and/or applied to the support or previously printed printing layers. Furthermore, the printing screen can have at least one cutout with sections that merge into one another.

In a further preferred manner, the cutouts of the printing screen and/or the printing stencil can correspond with predefined arrangement and/or receiving sections of the support. In this way, the printing medium can be printed specifically in and/or on the arrangement and/or receiving sections of the support. In particular, such an embodiment can prevent accidental or undesired printing of the support structure outside the arrangement and/or receiving sections. At the same time, a high printing volume per printing process can be realized.

According to a further preferred embodiment, at least one cutout of the printing screen and/or the printing stencil for the passage of the printing medium may have a diameter from 1 mm to 100 mm, in particular from 2 mm to 50 mm, preferably from 2 mm to 40 mm, preferably from 2 mm to 30 mm, more preferably from 3 mm to 30 mm, further preferably from 3 mm to 25 mm, further preferably from 3 mm to 20 mm, still further preferably from 4 mm to 20 mm, further preferably from 4 mm to 15 mm, further preferably from 4 mm to 10 mm, still further preferably from 4 mm to 9 mm, further preferably from 4 mm to 8 mm, still further preferably from 5 mm to 10 mm, further preferably from 5 mm to 8 mm. Likewise, at least one cutout of the printing screen and/or of the printing stencil for the passage of the printing medium can have a size corresponding to the preceding diameter values. This applies in particular to cutout shapes that deviate from a circular shape. With such a dimensioned cutout in the printing screen and/or in the printing stencil, on the one hand only a low mechanical stress on the living cells in the printing medium and at the same time a relatively large printing volume per printing process can be ensured.

According to a preferred embodiment, a plurality of surface, arrangement and/or receiving sections of the support can be printed onto and/or filled with the printing medium simultaneously and/or in one printing process and/or by a blading process. Similarly, different sections of a previously printed layer can be printed onto with the printing medium simultaneously and/or in one printing process and/or by one blading process. This can be accomplished with only little effort. In particular, all arrangement and/or receiving sections of the support or all sections of the previously printed layer can be printed onto with the printing medium in one printing process. Sequential printing of individual sections in many individual steps can thus be avoided.

It is also possible for different surface, arrangement and/or receiving sections of the support or different sections of a previously printed layer to be printed onto in chronological sequence according to their distance from one another and according to the respective printing blade speed. The individual arrangement and/or receiving sections of the support can thus be printed in accordance with a printing blade movement. Immediate simultaneous printing of the arrangement and/or receiving sections of the support or the sections of a previously printed layer can thus be omitted, which increases the process flexibility.

According to a further preferred embodiment of the method according to the invention, the printing medium can be printed at individual positions on a carrier structure. By printing the print medium onto a carrier structure printing sections that are independent of one another and/or fluid-technically separated can be generated in a particularly advantageous manner. As a result, several independent tissues, tissue structures, cell cultures and/or cell agglomerations can be generated, which are available for a relatively large number of medical treatments and/or pharmacological examinations.

According to a further preferred embodiment, different printing screens can be used for printing a tissue, in particular to create differently shaped printing layers. Furthermore, the different shaping of different printing screens can be used to create complex three-dimensional tissue structures, in particular blood vessels, vessel walls, channels, structural frameworks, matrices and/or scaffolds. In this way, a particularly advantageous reproduction of biological tissue structures can be realized.

In a further preferred manner, different printing media and/or printing media with different cells can be used to print different layers, in particular to produce tissue with different layer properties and/or to produce different skin layers. Furthermore, a variation of the printing medium can be carried out in a print build-up direction (z-direction). In this way, tissue structures with a high degree of functionality can be generated.

It can also be advantageous if a tissue variation is generated within a printing layer, in particular along an x/y direction and/or transverse to a print build-up direction. Furthermore, tissue variations can be generated by using different printing screens and/or printing stencils, in particular different printing screens and/or printing stencils per cell type and/or printing area. In this way, particularly detailed replicas of biological tissue structures can be generated.

It may be further advantageous to superimpose a sterile gas medium on the printing medium during printing, before printing and/or after printing, in particular sterile air. Sterility requirements for handling and/or processing living cells can be safely followed in this way. Unwanted contamination of the cell cultures and/or cell agglomerations and/or the printed and/or produced tissue can thus be avoided.

The printing medium can in particular be a nutrient medium and/or a medium containing nutrients, in particular a nutrient liquid. Such a printing medium or nutrient medium can be designed in particular as a so-called bio ink.

According to a still further preferred embodiment, the printing medium can be in the form of a printing paste and/or low-viscosity or medium-viscosity nutrient liquid and/or liquid suspension. In purely gel-like media, cells and/or nutrients can only diffuse to a limited extent, which can lead to the availability of nutrients being limited and thus also to the lifespan of the cells being limited. By forming the printing medium as a relatively low-viscosity printing paste and/or low-viscosity or medium-viscosity nutrient liquid and/or liquid suspension, a high degree of nutrient supply to the living cells can be ensured within the printing medium.

It is also possible for the printing medium to be in the form of a gel and/or a highly viscous nutrient medium, which can simplify processing or printing.

Furthermore, the printing medium can be designed as a sol-gel matrix and/or have a variable viscosity and/or the viscosity of the printing medium can be changed by a drying step and/or a tempering step, in particular a temperature reduction or temperature increase. Depending on the processing step performed, the viscosity can thus be changed. In this way, a high level of nutrient supply can be ensured over relatively long periods of time and, at the same time, the viscosity can be changed for the purpose of processing or further processing steps.

The printing medium can also be designed to be structurally viscous and/or shear-thinning. In the case of relatively high shear stresses on the printing medium, this can result in a temporarily lower viscosity and thus reduce the shear stress. This further reduces the risk of cell damage.

In a further preferred manner, a drying and/or tempering step of the printed printing medium can take place between successive printing steps. It is also possible that a sol-gel transition is generated after the printing of the printing medium, in particular by a drying and/or tempering step, and/or that a further printing process is carried out after a generated sol-gel transition. The properties of the printing medium or of the cell culture and/or cell agglomeration produced as a result can thus be specifically influenced as a function of the respective process stage.

In a further preferred manner, the printing medium can be liquid or have a low viscosity during printing, so that simple printing is possible. Following printing or printing of a printing layer, the respective printing layer can be solidified or the viscosity of the printed medium can be increased, for example, by tempering and/or by creating a sol-gel transition. The respective printed object or the cell culture and/or cell agglomeration generated by printing can thus be built up in a suitable manner in a layer build-up direction (z-direction).

As soon as the respective print object is built up in height (z-direction), it can be inserted into a suitable mold and/or stencil or the mold and/or stencil can be placed on the print object to surround it. The printed object can thus be limited in the x/y direction. The printed medium or the cell culture and/or cell agglomeration produced by printing can be returned to a more fluid state or to a state with lower viscosity, respectively, without melting. This allows nutrients to circulate freely again within the cell culture and/or cell agglomeration produced by printing. Tissue can develop in an improved manner.

In a further preferred embodiment, the printing medium may contain cells capable of division and/or cells inducible to division. Further, the living cells of the printing medium may be human, animal and/or plant cells. In particular, the printing medium may contain all types of human, animal and/or plant cells. Particularly preferably, the living cells may be all cells of the human or animal body or of plants that are capable of division or can be induced to divide. This results in a particular suitability for medical and pharmacological examinations.

In a further preferred manner, the printing medium may comprise living cells from the group of primary cells, in particular all types of human, animal and/or plant primary cells.

Similarly, the printing medium may have living cells from the group of cell lines, in particular all types of human, animal and/or plant established cell lines.

Primary cells may be, for example, organ cells, in particular organ cells of all organs, skin cells, fibroblasts, chondroblasts, osteoblasts, muscle cells, cardiac muscle cells, nerve cells, liver cells, islet cells, vascular cells, glandular tissue cells, tumor cells, in particular tumor cells of all tumor tissues, stem cells, in particular hematopoietic, mesenchymal and/or neuronal stem cells and/or induced pluripotent stem cells from tissues, such as fat tissue, skin and/or umbilical cord, and/or totipotent stem cells, in particular ovules and/or embryonic cells, and/or pluripotent, multipotent, oligopotent and/or unipotent stem cells, blood cells and/or immune cells.

Cell lines may be, for example, immortalized cells of the above-mentioned cell types and/or cells of the above-mentioned tissue types.

It may be further advantageous if the printing medium and/or the tissue produced contains different cells and/or cell types, in particular different cells and/or cell types as mentioned above. This can result in a further improved suitability of the produced cell cultures and/or cell agglomerations and/or tissue structures for medical and/or pharmacological examinations and/or treatments.

Further preferably, a tissue produced from the printing medium may contain different cells and/or cell types, in particular different cells and/or cell types as mentioned above. This can also result in a further improved suitability of the generated tissue for medical and/or pharmacological examinations.

It may be further advantageous if the printing medium contains at least one growth factor and/or at least one protein and/or extracellular matrix protein, in particular a growth factor and/or protein from the group of cytokines, in particular as growth regulators, interferons and/or interleukins, membrane components, laminins, collagens, in particular collagen type 4, proteoglycans, entactins, nidogens, cell adherence factors, in particular fibronectin and/or vitronectin, growth factors, in particular of the EGF family, the TGF family, PDGF, VEGF, somatomedins, in particular IGF, NGF, PTGF, and/or protective factors, in particular tissue-specific plasminogen activators, serum albumin and/or CMC. In a particularly preferred manner, the printing medium may comprise at least one or more of these substances from the aforementioned groups. This can result in further improved suitability of the cell cultures, cell agglomerations and/or tissue structures produced by means of the printing medium for carrying out medical and/or pharmacological examinations and/or treatments.

In a further preferred manner, the printing medium may have cells with a size of 5-50 μm, 5-40 μm, 10-50 μm, 10-40 μm, 10-30 μm, 20-40 μm, 25-40 μm, 25-30 μm in diameter or in cross-sectional length. A cell culture and/or a cell agglomeration and/or a tissue produced therefrom may have a number of at least 5 cells, in particular at least 10 cells, preferably at least 20 cells, in particular at least 50 cells, more preferably at least 100 cells, more preferably at least 200 cells or at least 300 cells. Furthermore, a cell culture and/or a cell agglomeration and/or a tissue produced therefrom may have a number of cells of up to 100,000,000 cells, of up to 10,000,000 cells, of up to 1,000,000 cells, of up to 100,000 cells, of up to 10,000 cells, of up to 1000 cells, in particular of up to 500 cells. In particular, a cell culture and/or a cell agglomeration and/or a tissue produced therefrom may have a number from 5 to 100.000.000 cells, from 5 to 10.000.000 cells, from 5 to 1.000.000 cells, from 5 to 10.000 cells, from 5 to 1000 cells, in particular from 10 to 1000 cells, 20 to 500 cells, 50 to 500 cells, 100 to 500 cells, 200 to 500 cells or 300 to 500 cells. In this way, particularly meaningful examinations can be carried out by means of the cell cultures and/or cell agglomerations produced by the printing medium, or tissue structures particularly suitable for medical treatments can be produced.

Another independent aspect of the present invention relates to a method for producing biological tissue, wherein a support for cellular tissue is provided, wherein a printing medium containing living cells is screen printed onto the support, and wherein the printed cells develop into tissue as a result of the printing and/or after the printing.

Another independent aspect of the present invention relates to a tissue and/or tissue structure, in particular an organ, produced by a method described above.

According to a particularly preferred embodiment, the cell cultures and/or cell agglomerations and/or tissue structures and/or tissue sections may be the same with respect to the metabolic state of the cells, the cell age and/or the number of cells and/or the dimension of the number of cells. Likewise, it is possible that in the cell cultures and/or cell agglomerations and/or tissue structures and/or tissue sections deviations with respect to the metabolic state of the cells, the cell age and/or cell number are less than 20%, in particular less than 10%, less than 5%, less than 3%, less than 2% or less than 1%. By such an embodiment, the cell cultures and/or cell agglomerations and/or tissue structures or tissue sections can be used for particularly far-reaching medical treatments.

Further embodiments of the present invention result from combinations of the features disclosed in the claims, the description and the figures. The present invention is explained below with reference to embodiments and associated drawings.

### BRIEF DESCRIPTION OF THE DRAWINGS

It is shown schematically in each case:

FIG. 1 a support for cell tissue and a printing screen or printing stencil arranged above it in perspective view,

FIG. 2 two tissues produced by a process according to the invention on a support according to FIG. 1 in perspective view.

### DETAILED DESCRIPTION

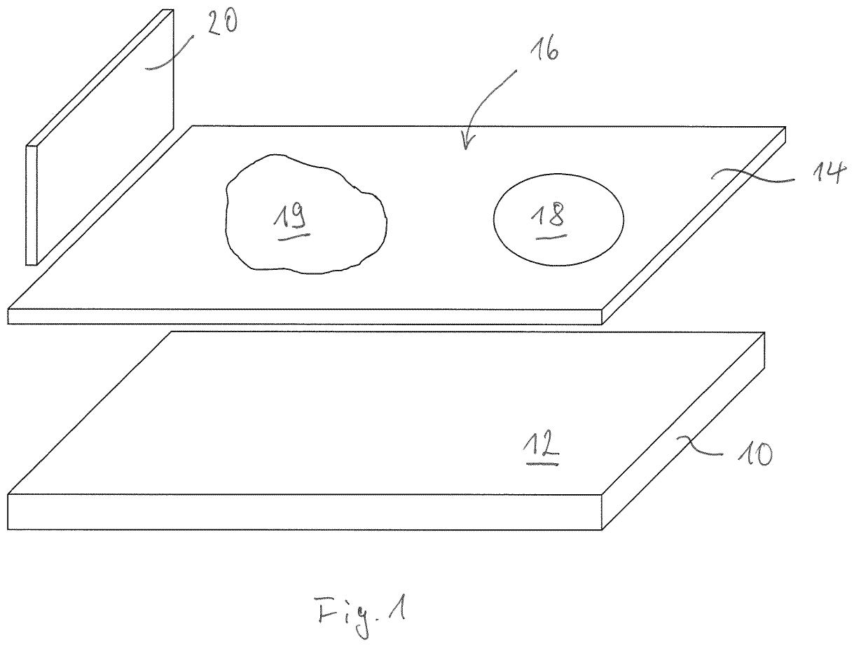

In FIG. 1, a support 10 for cell tissue, cell cultures and/or cell agglomerations is shown in perspective view. The support 10 can in particular be a machine-based support onto which a printing medium can be printed. Such a machine-based support 10 can be firmly connected to a printing system not shown here, in particular a 3D screen printing system. Likewise, the support 10 can be a carrier structure, a cell culture plate, an object carrier or a so-called array for cell tissue, cell cultures and/or cell agglomerations. Such a support can be positioned in a printing system for the respective printing operation or printing process and then removed from it again.

The support 10 may be suitable or configured for positioning, holding, and/or developing cell tissues, cell cultures, and/or cell agglomerations not shown in more detail herein. In particular, a printing medium containing living cells, not shown in greater detail herein, may be fed by printing onto a surface 12 of the support 10, as will be discussed in greater detail below. Once such printing medium is disposed on the surface 12, it may develop into cell tissue, cell cultures, and/or cell agglomerations. Similarly, a printing medium already consisting of cell cultures and/or cell agglomerations and/or containing cell cultures and/or cell agglomerations may be disposed on the surface 12 by printing and thereby develop into tissue. Accordingly, the printing medium may develop into cell tissue by printing and/or after printing.

FIG. 1 also shows a printing stencil 14 in schematic perspective view. The printing stencil 14 can be formed by a plate 16. Instead of the printing stencil 14, a printing screen not shown here can also be provided. The printing stencil 14 can have at least one cutout 18, 19 or also a plurality of cutouts 18, 19 for the passage of a printing medium. In the case of a plurality of cutouts 18, 19, these may in particular be formed separately from one another. The cutout 18 can, for example, have a diameter of from 1 mm to 100 mm, in particular from 2 mm to 50 mm, preferably from 2 mm to 40 mm, preferably from 2 mm to 30 mm, more preferably from 3 mm to 30 mm, more preferably from 3 mm to 25 mm, more preferably from 3 mm to 20 mm, even more preferably from 4 mm to 20 mm, more preferably from 4 mm to 15 mm, more preferably from 4 mm to 10 mm, even more preferably from 4 mm to 9 mm, more preferably from 4 mm to 8 mm, even more preferably from 5 mm to 10 mm, more preferably from 5 mm to 8 mm. The cutout 19 may have a size corresponding to the diameter-based size specifications mentioned above. The cutouts 18 and 19 may have different diameters and/or sizes or identical diameters and/or sizes.

In accordance with the present invention, a printing medium containing living cells is printed through a printing screen and/or through the printing stencil 14.

For printing the printing medium, a printing screen and/or the printing stencil 14 can be arranged above the support 10, as shown in FIG. 1. The printing blade 20 further shown schematically in FIG. 1 can be used for printing a printing medium through a printing screen and/or through the printing stencil 16. Thereby, the printing medium can be printed through a printing screen and/or the printing stencil 16 by at least one blading process and/or at least one printing blade movement, in particular by a plurality of blading operations of the printing blade 20 and/or a plurality of printing blade movements of the printing blade 20. In this way, the printing medium can be fed onto the support 10 in a particularly reliable manner.

Printing of the printing medium by means of a printing screen and/or by means of the printing stencil 14 and/or the printing blade 20 can be carried out in particular using a screen printing process. The screen printing process can in particular be a 2D screen printing process or 3D screen printing process.

It is also possible for the support 10, in particular when formed as a carrier structure, to be printed together and/or alternately with the printing medium containing living cells. In particular, the printing of the printing medium containing living cells can be carried out together and/or alternately with the printing of a support 10 formed as a carrier structure in a 3D screen printing process. Further, the support 10 may be printed prior to printing the printing medium containing living cells, in particular by 3D screen printing. Likewise, a support 10 formed as a carrier structure may be otherwise manufactured and provided prior to printing the printing medium containing living cells.

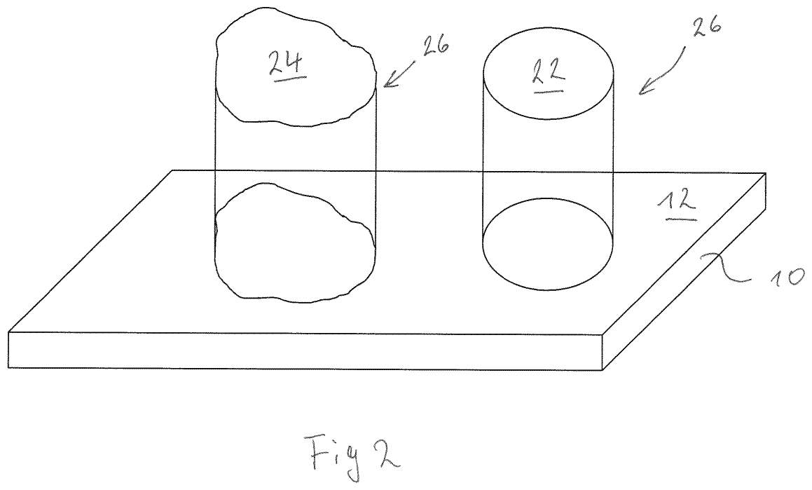

In FIG. 2, several tissues 22 and 24 are shown on a support 10 in perspective view. The tissues 22 and 24 are, in particular, biological cell tissues and tissue structures, respectively. The tissues 22 and 24 may be parts or sections of an organ, in particular parts or sections of a human or animal organ. Likewise, a complete organ may also be printed. Single or multi-layered tissues 22 and 24 may be produced by printing the printing medium, which is not shown in detail herein. Similarly, printing of the printing medium can create single or multi-layered and/or single or multi-layered cell agglomerations 24, which then develop into the tissues 22 and or 24.

Further, by printing the printing medium on the carrier structure 10, a three-dimensional cell culture and/or cell agglomeration can be generated. After printing the printing medium onto the carrier structure 10, the respective cell culture and/or cell agglomeration can develop into a three-dimensional tissue 22, 24.

It is also possible that structural matrixes and/or scaffolds are provided in printing sections 26 and tissues 22, 24, respectively, which are also not shown in more detail here. Such structural matrixes and/or scaffolds can be printed together with the printing medium and/or be contained therein.

In a particularly preferred manner, several sections of the surface 12 of the support 10 can be printed and/or filled with the printing medium simultaneously and/or in one printing process and/or by one blading process. Likewise, different sections of the surface 12 of the support 10 can be printed and/or filled in chronological sequence according to their distance from each other and according to the respective printing blade speed.

By printing the printing medium onto the support 10, printing sections 26 that are independent of one another and/or fluid-technically separate can be generated. In particular, one printing section 26 can be generated per surface section. A printing section 26 may form or develop into a tissue 22, 24 and/or a cell culture and/or a cell agglomeration. Further, the cell cultures and/or the cell agglomerations may develop into tissue after printing.

In the arrangement according to FIG. 2, the individual tissues 22, 24 and/or cell cultures and/or cell agglomerations may be the same with respect to the metabolic state of the cells, the cell age and/or the number of cells and/or the dimension of the number of cells. Likewise, it is possible that in the arrangement 11 according to FIG. 2 deviations with respect to the metabolic state of the cells, the cell age and/or the number of cells are less than 20%, in particular less than 10%, less than 5%, less than 3%, less than 2% or less than 1%.

The printing medium or the tissues 22, 24 and/or cell cultures and/or cell agglomerations produced therewith may contain cells capable of division and/or cells that can be induced to divide. The living cells of the printing medium and/or the tissues 22, 24 and/or cell cultures and/or cell agglomerations may be human, animal and/or plant cells. In particular, the printing medium and/or the tissues 22, 24 and/or cell cultures and/or cell agglomerations may contain all types of human, animal and/or plant cells. Particularly preferably, the living cells of the printing medium and/or the tissues 22, 24, the cell cultures and/or cell agglomerations can be all cells of the human or animal body or of plants that are capable of division or can be induced to divide.

Furthermore, the printing medium or a tissue 22, 24 produced therefrom and/or a cell culture and/or cell agglomeration produced from the printing medium may comprise living cells from the group of primary cells, in particular all types of human, animal and/or plant primary cells. Similarly, the printing medium or a tissue 22, 24 generated therefrom or a cell culture and/or cell agglomeration generated therefrom may comprise living cells from the group of cell lines, in particular all types of human, animal and/or plant established cell lines. Finally, the printing medium or a tissue 22, 24 generated therefrom or a cell culture and/or cell agglomeration generated therefrom may contain different cells and/or cell types.

Furthermore, the printing medium or a tissue 22, 24 produced therefrom or a cell culture and/or cell agglomeration produced therefrom may contain at least one growth factor and/or at least one protein and/or extracellular matrix protein, in particular a growth factor and/or protein from the group of cytokines, in particular as growth regulators, interferons and/or interleukins, membrane components, laminins, collagens, in particular collagen type 4, proteoglycans, entactins, nidogens, cell adherence factors, in particular fibronectin and/or vitronectin, growth factors, in particular of the EGF family, the TGF family, PDGF, VEGF, somatomedins, in particular IGF, NGF, PTGF, and/or protective factors, in particular tissue-specific plasminogen activators, serum albumin and/or CMC. Likewise, the printing medium or a tissue 22, 24 produced therefrom or a cell culture and/or cell agglomeration produced therefrom may comprise at least one or more of these substances from the aforementioned groups.

The tissues 22, 24 or cell cultures and/or cell agglomerations produced according to the methods described above are particularly preferably suitable for carrying out medical treatments and/or pharmacological examinations.

1. A method for producing biological tissue (22, 24) in which a support (10) for cell tissue (22, 24) is provided, in which a printing medium containing living cells is provided, in which the printing medium is printed onto the support (10) through a printing screen and/or a printing stencil (14), and in which the printed cells develop into tissue (22, 24) as a result of the printing and/or after the printing.

2. The method according to claim 1, wherein: the printing medium is printed through the printing screen and/or the printing stencil (14) onto the support (10) with or without a scaffold, a matrix and/or a structural framework and/or
the structural frameworks, matrices and/or scaffolds are provided on the support (10), and/or
the structural frameworks, matrices and/or scaffolds are printed together with and/or contained in the printing medium.

3. The method according to claim 1, wherein:
the printing medium is printed in layers and/or built up in layers, and/or
a single printing layer and/or printing thickness is produced in a single printing operation, and/or
a printing layer has a smaller thickness in the print build-up direction than the extent of the printing layer transverse to the print build-up direction.

4. The method according to claim 1, wherein the printing medium is conveyed through the printing screen and/or the printing stencil (14) and onto the support (10) by at least one blading process and/or at least one printing blade movement.

5. The method according to claim 1, wherein:
the printing medium is printed onto the support (10) by at least one screen printing process, and/or
the printing of the printing medium onto the support (10) is carried out using a screen printing process.

6. The method according to claim 1, wherein:
by printing the printing medium onto the support (10) a three-dimensional cell culture and/or a three-dimensional cell agglomeration is generated, and/or
after printing onto the support (10) the respective cell culture and/or cell agglomeration develops into a three-dimensional tissue (22, 24) and/or a three-dimensional tissue structure.

7. The method according to claim 1, wherein the printed printing medium and/or the printed cells and/or cell agglomerations by the printing or after the printing develop into organ tissue, and/or to a partial or complete organ, and/or consumable meat, and/or meat-containing food.

8. The method according to claim 1, wherein the printing screen and/or the printing stencil (14) has at least one cutout (18, 19) for the passage of the printing medium, and/or
the printing screen and/or the printing stencil (14) has a plurality of cutouts (18, 19) for the passage of the printing medium, wherein the plurality of cutouts (18, 19) are formed separately from one another, and/or
the printing screen and/or the printing stencil (14) has at least one cutout (18, 19) with sections merging into one another.

9. The method according to claim 1, wherein:
different printing screens and/or printing stencils (14) are used for printing a tissue (22, 24) in order to produce differently shaped printing layers, and/or
complex three-dimensional tissues (22, 24) and/or tissue structures are produced by the different shaping of different printing screens and/or printing stencils (14)

10. The method according to claim 1, wherein:
different printing media and/or printing media with different cells are used for printing different layers for producing tissue (22, 24) with different layer properties and/or for producing different skin layers, and/or
a variation of the printing medium is carried out in a printing build-up direction.

11. The method according to claim 1, wherein:
a tissue variation is generated within a printing layer an x/y direction and/or transversely to a printing build-up direction, and/or
tissue variations are generated by using different printing screens and/or printing stencils (14) per cell type and/or printing area.

12. The method according to claim 1, wherein: the printing medium is formed as printing paste and/or low-viscosity or medium-viscosity nutrient liquid and/or liquid suspension and/or sol-gel matrix, and/or
the printing medium has a variable viscosity, and/or
the viscosity of the printing medium can be changed by a drying step and/or a tempering step, and/or
the printing medium is structurally viscous.

13. The method according to claim 1, wherein: a drying and/or tempering step of the printed printing medium is carried out between successive printing steps, and/or
a sol-gel transition is generated after the printing of the printing medium by a drying and/or tempering step, and/or
a further printing process is carried out after a generated sol-gel transition.

14. The method according to claim 1, wherein a support (10) for cell tissue (22, 24) is provided, wherein a printing medium containing living cells is printed onto the support (10) by means of screen printing, and wherein the printed cells develop into tissue (22, 24) by the printing and/or after the printing.

15. An organ tissue (22, 24 produced by the method according to claim 1.

16. The method according to claim 7, wherein the organ tissue is selected from the group consisting of liver tissue, kidney tissue, heart tissue, skin tissue, muscle tissue, and combinations thereof.

17. The method according to claim 7, wherein the partial or complete organ is for transplantation and is optionally selected from liver, kidney, heart and/or skin.

18. The method according to claim 9, wherein the tissue is selected from the group consisting of blood vessels, vessel walls, channels, structural frameworks, matrices, and/or scaffolds.
